(19) United States
(12) Patent Application Publication
(10) Pub. No.: US 2018/0344227 A1
(43) Pub. Date: Dec. 6, 2018

(54) PULSE OXIMETRY SYSTEM WITH AN INTEGRATED PULSE WIDTH MODULATOR

(71) Applicant: KONINKLIJKE PHILIPS N.V., EINDHOVEN (NL)

(72) Inventors: John CRONIN, BONITA SPRINGS, FL (US); Michael D'ANDREA, BONITA SPRINGS, FL (US)

(21) Appl. No.: 15/778,829

(22) PCT Filed: Dec. 1, 2016
(86) PCT No.: PCT/EP2016/079391

§ 371 Date: May 24, 2018

Related U.S. Application Data
(60) Provisional application No. 62/261,315, filed on Dec. 1, 2015.

(30) Foreign Application Priority Data
Mar. 17, 2016 (EP) 16160930.0

(51) Int. Cl. A61B 5/1455; A61B 5/1495; A61B 5/00

(52) U.S. Cl. CPC A61B 5/14551; A61B 5/1495; A61B 5/7221; A61B 5/6826; A61B 2560/0238; A61B 5/7228

(57) ABSTRACT

A system and method comprising a pulse oximeter with a pulse width modulator that periodically tests the en wavelengths emitted at the pulse width's upper and lower limits in order to ensure that the median pulse width is emitting the wavelength specified by the manufacturer. The system comprises a pulse oximeter that has LED emitter(s), photodetectors, a pulse width modulator and memory. The specifications database contains data relating to the wavelength specified by the manufacturer. The calibration software will periodically test the LED to ensure the correct wavelength is being emitted by the LED in order to detect the proper constituents in the patient's blood. The testing is conducted whenever the pulse oximeter is energized and then again at a specified interval until power is disconnected from the pulse oximeter. Each test includes increasing the pulse width until an error is detected, recording the upper limit of the pulse width and the corresponding wavelength before an error occurred. Then performing the same test by decreasing the pulse width until an error is detected. The wavelength emitter at the median pulse width is then calculated and compared to the manufacturer's specification. If they do not match the calibration software adjusts the pulse width until the manufacturer's specification is achieved.

### BACKGROUND OF THE INVENTION

The present invention is directed to a system and method for calibrating pulse oximetry pulse width and wavelength parameters using pulse width modulation. U.S. Pat. No. 5,784,151 discloses a system that comprises a pulse width modulation (PWM) module 39 which is operated by CPU. This patent discloses an apparatus for testing a pulsed light oximeter and a light sensing means for producing an electrical pulse signal representative of light flashes emitted by the oximeter, each light flash emitted having one of a plurality of predefined wavelengths. U.S. Pat. No. 5,577,500 discloses a pulse width modulator and a means to control the peak-to-peak values of both the red and infrared signals so that they extend across most of the range of the A/D converter to give maximum resolution. U.S. patent application number 2005/0187450 discloses a pulse oximeter comprising a controller for generating a pulse width modulator (PWM) drive signal to said LED and a system configured to determine if a forward voltage of said LED is within a predetermined range using a measurement of said current and said PWM signal.

### SUMMARY OF THE CLAIMED INVENTION

The present invention relates to pulse oximeters with a pulse width modulator that periodically tests the wavelengths emitted at the pulse width's upper and lower limits in order to ensure that the median pulse width is emitting the wavelength which has been specified. In one embodiment the wavelength is specified by the manufacturer of the pulse oximeter. The system comprises a pulse oximeter that has LED emitter(s), photodetectors, a pulse width modulator and memory. On the memory is calibration software, a testing database and a specifications database. The specifications database contains data relating to the wavelength specified by, for example, the manufacturer. The calibration software will periodically test the LED to ensure the correct wavelength is being emitted by the LED in order to detect the proper constituents in the patient's blood. The testing is conducted whenever the pulse oximeter is energized and then again at a specified interval until power is disconnected from the pulse oximeter. Each test consists of increasing the pulse width until an error is detected, recording the upper limit of the pulse width and the corresponding wavelength before an error occurred. Then performing the same test by decreasing the pulse width until an error is detected. The wavelength emitter at the median pulse width is then calculated and compared to the given, or desired, specification. If they do not match the calibration software adjusts the pulse width until the desired specification is achieved.

### BRIEF DESCRIPTION OF THE DRAWINGS

The accompanying drawings, which are included to provide a further understanding of the invention, are incorporated herein to illustrate embodiments of the invention. Along with the description, they also serve to explain the principle of the invention. In the drawings:

FIG. 1 illustrates a pulse oximetry system according to the present invention comprising an LED tuner integrated into a pulse oximeter.

FIG. 2 illustrates a pulse oximeter system comprising a pulse width modulator and a memory according to a preferred embodiment of the invention.

FIG. 3 is a flowchart illustrating a process according to a preferred embodiment of the invention.

FIG. 4 illustrates a database comprising various parameters used for adjusting the pulse width and wavelength according to a preferred embodiment of the present invention.

### DETAILED DESCRIPTION OF THE EMBODIMENTS

The following are definitions of terms as used in the various embodiments of the present invention.

The term “database” as used herein refers to a collection of data and information organized in such a way as to allow the data and information to be stored, retrieved, updated, and manipulated and to allow them to be presented into one or more formats such as in table form or to be grouped into text, numbers, images, and audio data. The database typically resides in computer memory that includes various types of volatile and non-volatile computer memory. “Database” as used herein also refers to conventional databases that may reside locally or that may be accessed from a remote location, e.g., remote network servers. The term “database” as used herein may also refer to a segment or portion of a larger database, which in this case forms a type of database within a database. Memory wherein the database resides may include high-speed random access memory or non-volatile memory such as magnetic disk storage devices, optical storage devices, and flash memory. Memory where the database resides may also comprise one or more software for processing and organizing data received by and stored into the database.

The present invention is directed to a system comprising a pulse oximeter that has LED emitter(s), photodetectors, a pulse width modulator, and memory. On the memory is calibration software, a testing database and a specifications database. The specifications database includes data specifying the wavelength specified by the manufacturer (e.g., 660 nm). The calibration software periodically tests the LED to ensure that the specified wavelength is being emitted by the LED in order to ensure accurate pulse oximetry measurements. The testing is conducted when the pulse oximeter is enabled to take measurements, and preferably at specified intervals (e.g., every 5 minutes) until power is disconnected from the pulse oximeter. Each calibration test comprises increasing the pulse width until an error is detected, recording the upper limit of the pulse width and the corresponding mean wavelength before an error is detected. Then, the calibration test is perform again this time by decreasing the pulse width until an error is detected. The median pulse width is calculated and compared to the manufacturer's specification. If they do not match, the calibration software adjusts the pulse width until the manufacturer's specification is matched.

In a preferred embodiment, the wavelength emitted by an LED in a pulse oximeter is tuned in order to detect a second variable. A pulse modulation software is used to regulate the pulse width. Because the pulse width can change over time due to factors such as LED degradation, constantly calibrating the mean wavelength for a range of pulse width values, the system of the present invention can ensure that the LED is outputting 660 nm, instead of 664 or 657 nm, for example.

In one embodiment of the present invention, the median wavelength is measured to be 970 nm, which is the wavelength corresponding to O2 bound to iron. If the PWM is modulated around a reference value and the LED is presently outputting 964 to 974 nm, which are all being absorbed but at 963 nm the system gets an error reading and at 975 nm, the system detects another error reading, the median and range that does not give rise to an error reading can thus be calculated. After, e.g., 2 hours from the time the pulse oximetry measurement was initiated, the LED's median wavelength was observed to drift and it is outputting, at the same PWM, say 962 nm, this means the signal being measured at that wavelength is incorrect. So, every now and then while performing “ranging,” it is found that, prior to the detection of the error, the median wavelength has moved up to 975 nm (or the PWM has moved up), we know get 969 to 980 nm as the range where 975 is the median. This means the median PWM should be changed to match the median reading of O2 attached to the iron. This allows the necessary correction to be applied to the PWM signal, which was initially believed to still be at the reference value but was in fact no longer corresponds to the median wavelength, e.g., the PWM for 970 nm is really the PWM for 962 nm.

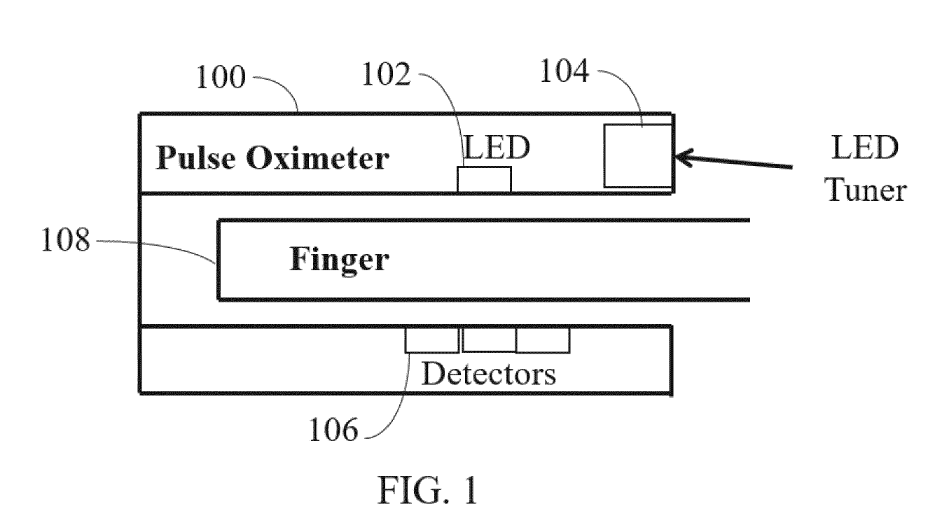

FIG. 1 shows a preferred embodiment of the invention that integrates an LED tuner 104 into a pulse oximeter 100. In the figure, the patient's finger 108 is inside the pulse oximeter between the LED 102 and light detectors 106.

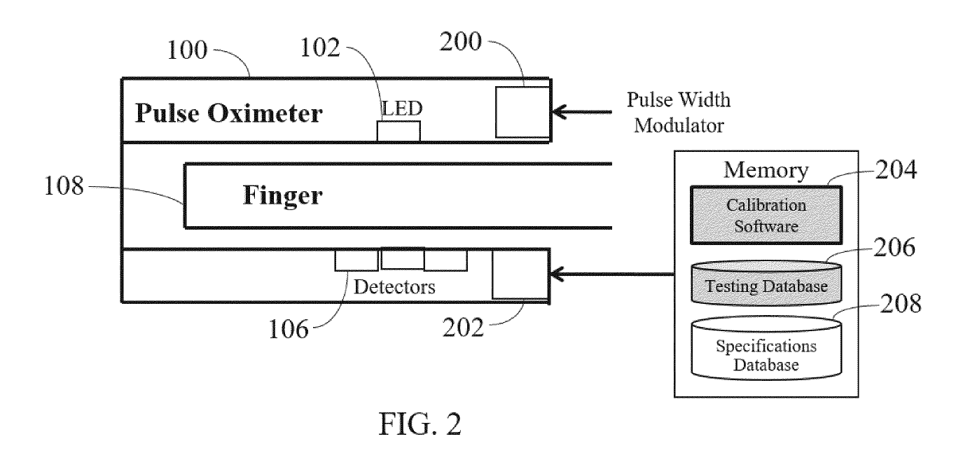

FIG. 2 shows a pulse oximeter 100, LED 102, with a PWM (pulse width modulator) 200, detectors 106 and memory 202 wherein the calibration software 204, testing database 206, and specifications database 208 reside.

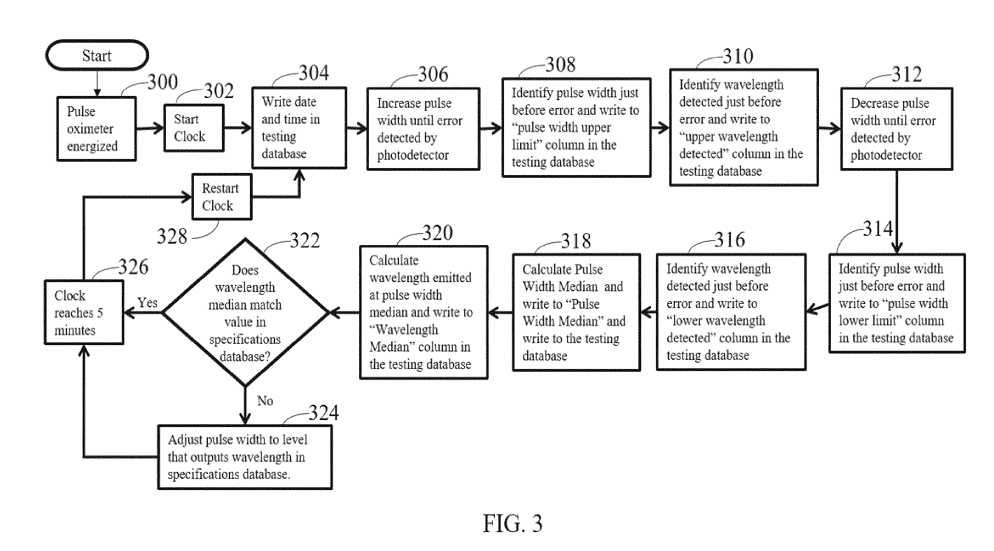

FIG. 3 is a flowchart that illustrates a preferred embodiment of the present invention. Here, the software module is triggered when the pulse oximeter is connected to a power source (step 300). The software starts a clock (step 302) and writes the date and time in the testing database (step 304). The pulse width is then increased until an error is detected by the photodetector (step 306). The highest pulse width achieved before the error occurred and the corresponding wavelength emitted are recorded in the “pulse width upper limit” (step 308) and “upper wavelength detect” columns (step 310) in the testing database. The same process is then repeated in reverse to detect the lower limit of the pulse width before an error occurs (step 312). Then recording the pulse width and corresponding wavelength emitted in the “pulse width lower limit” (step 314) and “lower wavelength detect” (step 316) columns in the testing database. The mean pulse width (step 318) and the corresponding wavelength emitted (step 320) is then calculated and the values are written to the “pulse width median” (step 318) and “wavelength median” columns (step 320) in the testing database. The wavelength emitted at the pulse width median in then compared to the manufacturer's specification in the specification database (step 322). If the values match and the clock has reached a specified interval, such as 5 minutes, the clock is restarted and the testing process is repeated (step 326). If the values do not match, the pulse width is adjusted to output the manufacturer's specified wavelength (step 324). Once the clock reaches a specified interval, such as 5 minutes, the clock is restarted and the testing process begins again.

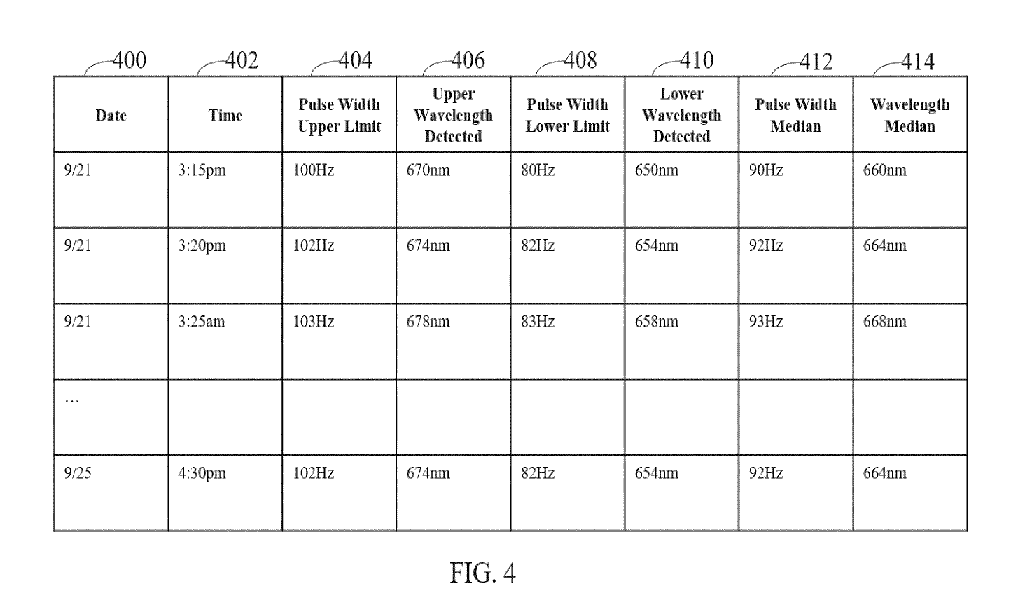

FIG. 4 shows a database according to a preferred embodiment of the invention, along with example data. The database comprises several columns of data corresponding to different parameters: “Date” 400, “Time” 402, “Pulse Width Upper Limit” 404, “Upper Wavelength Detected” 406, “Pulse Width Lower Limit” 408, “Lower Wavelength Detected” 410, “Pulse Width Median” 412, and “Wavelength Median” 414. The database is located in the memory in the pulse oximeter. The database is where the result of each test completed by the calibration software is stored into. The first two columns house the date 400 and time 402 of each test. The next two columns have the pulse width upper limit 404 achieved during the test before encountering an error and the corresponding wavelength emitted 406 at that pulse width upper limit. The next two columns have the pulse width lower limit 408 achieved during the test before encountering an error and the corresponding wavelength emitted 410 at that pulse width lower limit. The final two columns have the calculated median values for both pulse width 412 and wavelength between their upper and lower limits 414.

The present invention is not intended to be restricted to the several exemplary embodiments of the invention described above. Other variations that may be envisioned by those skilled in the art are intended to fall within the disclosure.

1. A method for calibrating pulse width and median wavelength of a pulse oximetry system, the method comprising:
increasing a width of a pulse until an error reading is detected;
recording an upper limit of the pulse width and a corresponding mean wavelength before the error reading is detected;
decreasing the pulse width until another error reading is detected;
calculating a median pulse width;
comparing the calculated median pulse width with a given specification of the pulse oximetry system, wherein the comparison reveals a mismatch between the calculated median pulse width and the given specification; and
adjusting the pulse width using a calibration software until the given specification is matched.

2. The method of claim 1, wherein the error reading detected is a photodetector error reading.

3. The method of claim 2, wherein the photodetector error reading arises from a detected wavelength outside of the given specification.

4. The method of claim 2, wherein the photodetector error reading arises from a substantially flat reading over a given specified wavelength range being detected.

5. The method of claim 1, wherein the pulse is emitted by a light-emitting device (LED) of the pulse oximetry system.

6. The method of claim 1, further comprising recording a lower limit of the pulse width and a corresponding mean wavelength before the other error reading is detected.

7. A system for calibrating pulse width and median wavelength of a pulse oximetry system, the system comprising:
a pulse width modulator that increases a width of a pulse until an error reading is detected and that decreases the pulse width until another error reading is detected;
memory that records an upper limit of the pulse width and a corresponding mean wavelength before the error reading is detected;
a processor that executes instructions stored in memory, wherein execution of the instruction by the processor:
calculates a median pulse width,
compares the calculated median pulse width with a given specification of the pulse oximetry system, wherein the comparison reveals a mismatch between the calculated median pulse width and the given specification, and
adjusts the pulse width using a calibration software until the given specification is matched.

8. The system of claim 7, wherein the error reading detected is a photodetector error reading.

9. The system of claim 8, wherein the photodetector error reading arises from a detected wavelength outside of the given specification.

10. The system of claim 8, wherein the photodetector error reading arises from a substantially flat reading over a manufacturer specified wavelength range being detected.

11. The system of claim 7, wherein the pulse is emitted by a light-emitting device (LED) of the pulse oximetry system.

12. The system of claim 7, wherein memory further records a lower limit of the pulse width and a corresponding mean wavelength before the other error reading is detected.

13. A non-transitory computer-readable storage medium, having embodied thereon a program executable by a processor to perform a method for calibrating pulse width and median wavelength of a pulse oximetry system, the method comprising:
increasing a width of a pulse until an error reading is detected;
recording an upper limit of the pulse width and a corresponding mean wavelength before the reading error is detected;
decreasing the pulse width until another error reading is detected;
calculating a median pulse width;
comparing the calculated median pulse width with a given specification of the pulse oximetry system, wherein the comparison reveals a mismatch between the calculated median pulse width and the given specification; and
adjusting the pulse width using a calibration software until the given specification is matched.
